(19) United States
(12) Patent Application Publication
(10) Pub. No.: US 2005/0101968 A1
(43) Pub. Date: May 12, 2005

(54) Ostial locator device and methods for transluminal interventions

(76) Inventor: Daniel G. Dadourian, Byrn Mawr, PA (US)

Correspondence Address: COHEN SAKAGUCHI & ENGLISH LLP, 2040 MAIN STREET, 9TH FLOOR, IRVINE, CA, 92614, US

(21) Appl. No.: 10/712,888

(22) Filed: Nov. 12, 2003

(51) Int. Cl. A61F 11/00

(52) U.S. Cl. 606/108

(57) ABSTRACT

Apparatus and methods are provided for locating an interventional device relative to the ostium of a branch vessel wherein an ostial locator wire is attached to the interventional device so that a selectively deployable expandable section of the ostial locator wire encircles the interventional device. A diameter of the expandable section of the distal region is larger than the diameter of the ostium of the branch vessel, so that the expandable section flattens out as it is urged into contact with tissue surrounding the ostium of the branch vessel.

### FIELD OF THE INVENTION

The present invention relates to methods and apparatus for locating the ostium of a vessel, and more particularly, to methods and apparatus for positioning an interventional device relative to the ostium of a branch vessel.

### BACKGROUND OF THE INVENTION

Coronary artery stents were developed to address problems associated with conventional angioplasty, especially post-procedure narrowing of the vessel, referred to as “restenosis.” Conventional stents are substantially tubular structural supports that are positioned within a vessel to restore or maintain sufficient blood flow through the vessel.

Previously known methods of stent delivery involve introducing a non-deployed stent into a vessel, positioning the stent adjacent a treatment area within the vessel and deploying the stent to an expanded state to maintain the patency of the vessel.

It is often difficult to precisely locate the ostium of a vessel because a fluoroscope provides the clinician only a two dimensional view of the patient's three-dimensional anatomy. Consequently, when it is desired to place a stent at a lesion near the ostium of a main and branch vessel, it is not uncommon for the stent to be deployed too far into the branch vessel or conversely to extend through the ostium and into the main vessel.

FIG. 1 depicts a previously known method of maneuvering a stent, stent S, through main vessel MV to position the stent within branch vessel BV. As shown in FIG. 1, the stent is positioned too far from ostium O and into the branch vessel, so that it is offset slightly with respect to lesion L. As a result, the lesion may tend to occlude the proximal end of the stent, nearest to ostium O. In essence, the original blockage remains untreated and potentially threatens the stent due to a higher risk of acute closure.

FIG. 2 depicts the converse situation in which stent S is insufficiently advanced through ostium O and into the branch vessel BV. In this case, the proximal end of the stent extends into main vessel MV, thus possibly complicating future access to the branch vessel and serving as a site for the formation of thrombus.

U.S. Pat. No. 5,749,890 to Shaknovich describes a stent delivery catheter having a break segment disposed near its distal end. The delivery catheter includes a balloon or mechanical arrangement to selectively expand the diameter of the delivery catheter in the vicinity of the break segment. Once expanded, the diameter of the break segment is too large to enter the branch vessel, and thus abuts against the ostium of the branch vessel, thereby locating the stent at a desired pre-determined depth within the branch vessel.

One drawback of the system disclosed in the Shaknovich patent is that the distance between the location of the stent and the location of the break segment of the delivery catheter is fixed and pre-determined during manufacture of the stent and delivery catheter. Moreover, because the break segment forms a part of the stent delivery catheter itself, the clinician is necessarily limited in the selection and type of stent that can be used for a given patient and application.

In addition, the separate spherical balloon or mechanical arrangement employed in the break segment employed of the Shaknovich device to provide visual and mechanical feedback regarding the ostium may impede the clinician's ability to determine the true direction of orientation of the branch vessel from the main vessel in three dimensions.

In view of the foregoing drawbacks of previously known devices and methods, it would be desirable to provide methods and apparatus for locating the ostium of a vessel that can be used in conjunction with any commercially available interventional device, such as a guidewire, distal protection device, diagnostic catheter (such as ultrasound catheter), angioplasty or other treatment catheter or stent delivery catheter. Hereinafter, all such devices are collectively referred to as “interventional devices.”

It further would be desirable to provide methods and apparatus for precisely locating an interventional device relative to the ostium of a vessel that permit reduced use of contrast to visualize placement of a device within the branch vessel.

It still further would be desirable to provide methods and apparatus for precisely locating an interventional device relative to the ostium of a vessel that may be used with a wide variety of interventional devices, permit reduced use of contrast to visualize placement of a device within the branch vessel, and provide the clinician with tactile feedback regarding the distal end of the interventional device.

It also would be desirable to provide methods and apparatus for precisely locating an interventional device relative to the ostium of a vessel that permit the ostial locator to be selected responsive to the specific treatment or application.

### SUMMARY OF THE INVENTION

In view of the foregoing, it is an object of the present invention to provide methods and apparatus for locating an interventional device relative to the ostium of a vessel that can be used in conjunction with any commercially available guidewire, distal protection device, diagnostic catheter (such as ultrasound catheter), angioplasty or other treatment catheter or stent delivery catheter.

It is another object of this invention to provide methods and apparatus for precisely locating an interventional device relative to the ostium of a vessel that permit reduced use of contrast to visualize placement of a device within the branch vessel.

It is a further object of this invention to provide methods and apparatus for precisely locating an interventional device relative to the ostium of a vessel that may be used with a wide variety of interventional devices, permit reduced use of contrast to visualize placement of a device within the branch vessel, and provide the clinician with tactile feedback regarding the distal end of the interventional device.

It is yet another object of the present invention to provide methods and apparatus for precisely locating an interventional device relative to the ostium of a vessel that permit the ostial locator to be selected responsive to the specific treatment or application.

These and other objects of the present invention are accomplished by providing apparatus for locating an interventional device relative to the ostium of a vessel comprising an ostial locator device having a locator wire that may be selectively advanced to determine the position of an ostium between a main vessel and branch vessel. In accordance with the principles of the present invention, the ostial locator device comprises a small diameter sheath having a distal region configured to be coupled to a shaft of a conventional interventional device. A locator wire is slidably received within a lumen of the sheath and includes a distal region that may be deployed from a straight configuration, when retracted within the sheath, to an extended, expanded configuration (hereinafter, the portion of the distal region that assumes the expanded configuration is referred to as the “expanded section”).

When deployed to the expanded configuration, the distal region of the locator wire preferably encircles a desired portion of the interventional device. The expanded section of the distal region may take on the form of a coil, sphere, disk, cone, amphora, petalled-arrangement or other suitable shape. Because the diameter of the distal region in the expanded section is larger than the diameter of the ostium of the branch vessel, the distal region flattens out when it abuts the tissue surrounding the ostium of the branch vessel, thereby providing the clinician with visual and tactile feedback regarding the position of the distal region of the locator wire and attached interventional device. The exact three-dimensional directional orientation of the branch vessel from the main vessel is similarly identified. Once the ostium has been located, e.g., a stent may be deployed in proper alignment with the branch vessel area near the ostium.

Another aspect of the present invention involves a method of locating the ostium of a branch vessel including steps of providing an interventional device and attaching an ostial locator device thereto so that a distal region of a locator wire is arranged to substantially encircle a desired portion of the interventional device. The ostial locator device and interventional device then are advanced together through a main vessel and into a branch vessel to the vicinity of the ostium.

Once in the vicinity of the main vessel/branch vessel ostium, a distal region of the locator wire is deployed to its expanded configuration, wherein the distal region encircles a desired portion of the interventional device, and the devices are advanced together until the distal region of the locator wire abuts against the ostium of the branch vessel. The interventional device then may be used for its intended purpose at a position determined by operation of the ostial locator device.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a side-sectional view depicting a stent deployed in a branch vessel using previously known methods and apparatus;

FIG. 2 is a side-sectional view of an alternative depiction of a stent deployed in a branch vessel using previously known methods and apparatus;

FIG. 3 is a perspective view of an exemplary ostial locator device constructed in accordance with the principles of the present invention;

FIG. 4 is a perspective view of the ostial locator device of FIG. 3 coupled to a conventional stent delivery catheter;

FIG. 5 is a perspective view of an alternative fastener suitable for coupling the ostial locator device of the present invention to an interventional device;

FIGS. 6A-6D are side-sectional views depicting a method of using the ostial locator device of the present invention to properly align a stent with the ostium of a branch vessel; and

FIGS. 7A-7F are perspective views of alternative embodiments of the distal region of an ostial locator wire, in the expanded configuration, constructed in accordance with the principles of the present invention.

### DETAILED DESCRIPTION OF THE INVENTION

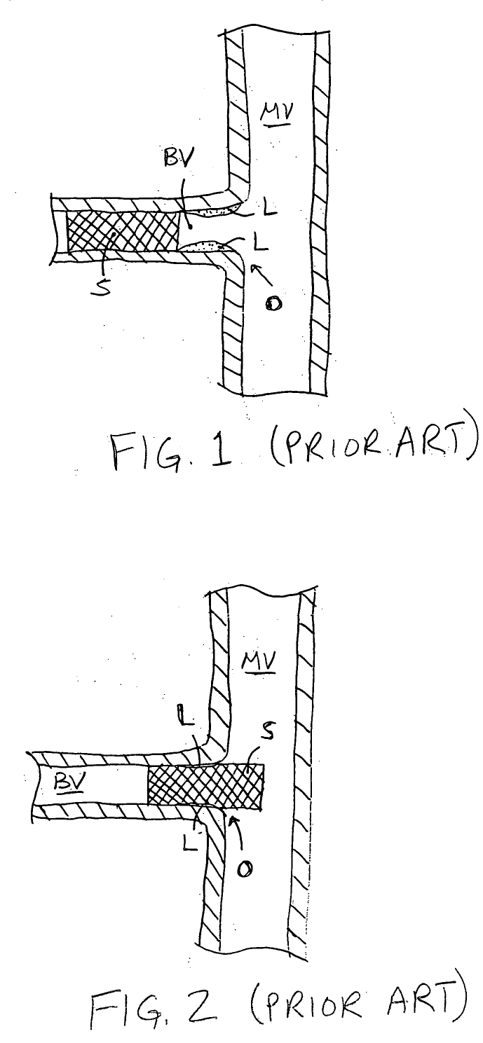

As described hereinabove, previously known methods and apparatus for deploying an interventional device within a branch vessel may lead to some misalignment between the actual deployment position and the preferred deployment position. This difference often results from the artifacts that occur when attempting to position an interventional device in a three dimensional space using the two-dimensional view provided by a fluoroscope. Consequently, for example, a stent may be deployed either too far into the branch vessel as in FIG. 1, or not far enough into the branch vessel, as in FIG. 2. Previous attempts to address these drawbacks have resulted in the development of stent or procedure specific delivery devices, thus limiting the availability and applicability of such prior art attempts to address the ostial location problem.

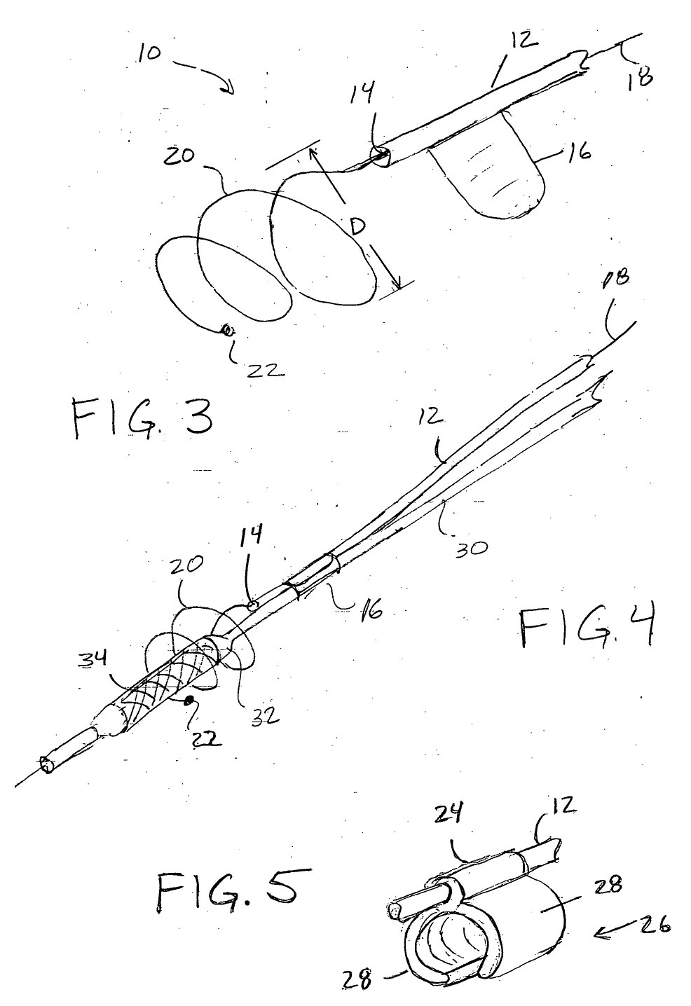

Referring now to FIGS. 3 and 4, a first exemplary embodiment of ostial locator device 10 constructed in accordance with the principles of the present invention is described. Ostial locator device 10 comprises elongate sheath 12 having lumen 14 and fastener 16. Locator wire 18 is disposed within lumen 14 and has distal region 20 that assumes an expanded configuration when deployed from the distal end of sheath 12.

Sheath 12 preferably comprises a flexible, high strength material, such as polyethylene or polyurethane, and has a length of between 60 to 120 cm, so that the proximal end of the sheath will extend outside the patient's body and may be manipulated by a clinician. Fastener 16 illustratively comprises a thin flexible sheet carrying a biocompatible adhesive, and permits ostial locator device 10 to be coupled to an interventional device. As shown in FIG. 4, the sheet of fastener 16 is wrapped around shaft 30 of the interventional device, illustratively, a stent delivery catheter, with sheath 12 proximal of balloon 32 and stent 34. Alternatively, the sheet of fastener 16 may have a natural inwardly directed spring, so that it grippingly encircles the shaft of delivery catheter 30.

Locator wire 18 preferably comprises a shape-memory material, such as a nickel-titanium alloy. Locator wire 18 is manufactured using known techniques so that distal region 20 assumes a straight configuration when retracted within lumen 14 of sheath 12, and an expanded configuration when extended from lumen 14, as illustrated in FIG. 4. In the embodiments of FIGS. 3-6, distal region 20 illustratively assumes a spiral shape, although any other suitable shape may be employed, including sphere, cone, coil, disk, amphora, petalled-arrangement, etc. A proximal end of ostial locator wire 18 may include a stop so that ostial locator wire 18 is not extended from sheath 12 more than a distance needed to fully deploy distal region 20.

Preferably, the maximum diameter D of distal region 20 (see FIG. 3) in the expanded section is two to three times the diameter of the interventional device, so that the distal region encircles the interventional device when deployed. In FIG. 4, the spiral of distal region 20 encircles stent 34 mounted on balloon 32. Ostial locator wire 18 preferably has a diameter of about 0.014 inches, and optionally may include a hydrophilic coating. The distal end of locator wire 18 also may include atraumatic tip 22, e.g., a bead, to prevent injury to the vessel wall.

In accordance with the principles of the present invention, the maximum diameter D of the distal region in the expanded configuration, when deployed from sheath 12, is greater than the diameter of the ostium of the branch vessel with which the ostial locator wire is to be used. Because the diameter of the expanded section of distal region 20 is larger than the diameter of the ostium of the branch vessel, the distal region flattens out when it abuts the tissue surrounding the ostium of the branch vessel. This flattening out of the distal region provides the clinician with tactile and visual feedback regarding the position of the distal region of the locator wire and attached interventional device. Once the ostium has been located, the interventional device may be properly aligned with the branch vessel area precisely at the ostium.

In accordance with another aspect of the present invention, when in the expanded configuration, the first few turns of distal region 20 preferably assume a diameter only slightly larger than the diameter of the shaft of the interventional device encircled by distal region 20. This ensures that the distal region remains centered about the interventional device as the distal region abuts against the ostium of the branch vessel.

In accordance with another aspect of the present invention, the position at which the ostial locator device is attached to the shaft of the interventional device may be measured by the clinician so as to ensure that, when the expanded section of distal region 20 is abutted against the tissue surrounding the ostium, a desired portion of the interventional device is properly positioned within the branch vessel. Advantageously, the ostial locator device of the present invention permits any desired distance between the desired portion of the interventional device and ostium to be achieved based on the position at which the sheath is affixed to the interventional device.

In accordance with yet another aspect of the present invention, a clinician may maintain a stock of ostial locator devices having distal regions that deploy to different pre-set diameters, so that an ostial locator device having an expanded section (e.g., with respect to shape and maximum diameter) appropriate for the branch vessel ostium size may be selected for a given application. In addition, distal region 20 may comprise a radiopaque feature, e.g., a thin layer of gold, to enhance visibility of the expanded section under fluoroscopic examination.

Referring now to FIG. 5, an alternative embodiment of sheath 12 is described. In the embodiment of FIG. 5, sheath 12 includes fastener 24 in the form of clasp 26 having resilient prongs 28. Clasp 26 preferably comprises a high strength resilient material that allows prongs 28 to be snap-fit or friction-fit on to the shaft of an interventional device to affix ostial locator device 10 thereto. Prongs 28 preferably are sufficiently flexible so that interventional devices having different diameters may be fastened to sheath 12 using clasp 26. Alternatively, clasp 26 may include a biocompatible adhesive on its interior surface to ensure that there is no relative movement between sheath 12 and the shaft of the interventional device, once the clinician has affixed the sheath to the shaft at a desired position. As will be apparent to one of skill in the art of catheter design, alternative fasteners may be employed to affix the ostial locator device to an interventional device without departing from the scope of the present invention.

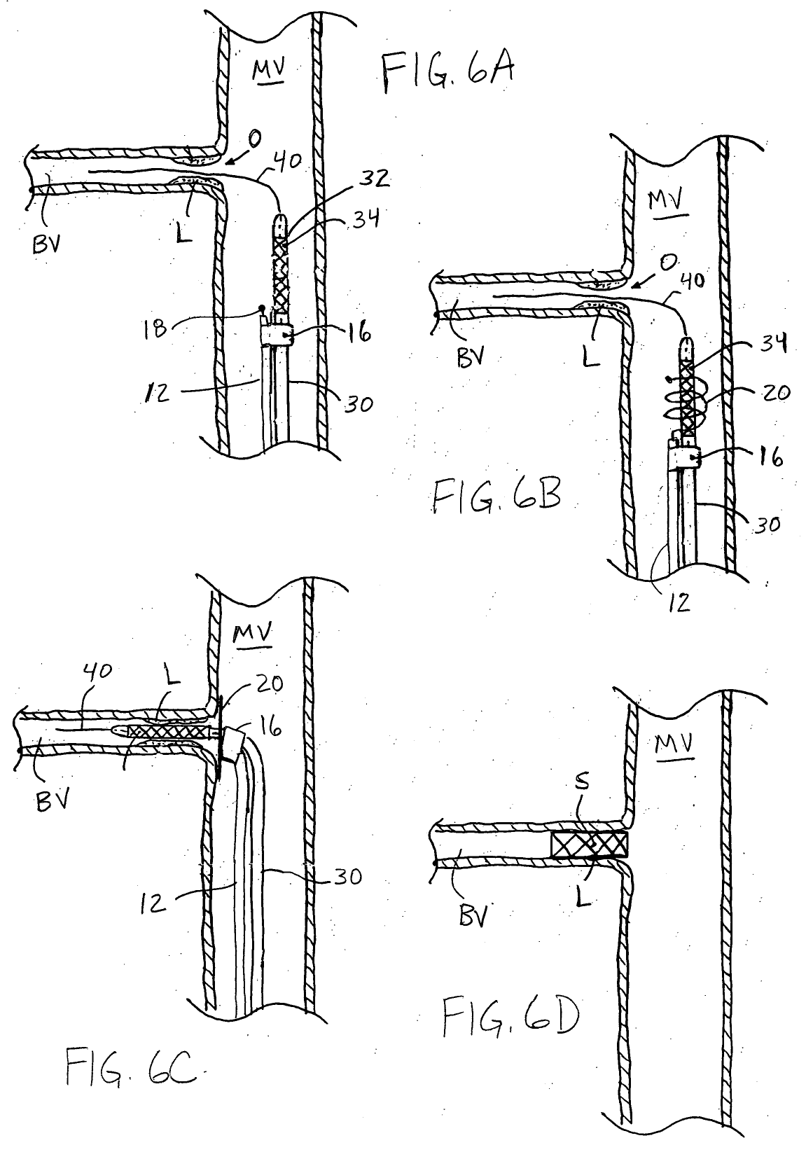

Referring to FIGS. 6A-6C, a method of using the ostial locator device of the present invention in a branched vessel is now described. Branch vessel BV includes a target treatment area having lesion L that causes a restriction of the branch vessel. In this case, the clinician desires to accurately deploy a stent over lesion L to restore the patency of the vessel.

In FIG. 6A, conventional guidewire 40 is advanced by a clinician through main vessel MV (e.g., the aorta) using a commercially available standard guide catheter that is selected by the clinician for the specific anatomical features expected to be encountered during the procedure. Guidewire 40 is advanced until the distal end of the guidewire is maneuvered into branch vessel BV through ostium O. Catheter 30 of FIG. 4, with ostial locator device 12 affixed thereto at a predetermined location, then is percutaneously advanced along guidewire 40 to a position adjacent to the ostium of the branch vessel.

Once the catheter and ostial locator device are positioned as shown in FIG. 6A, ostial locator wire 18 is advanced so that distal region 20 extends from sheath 12 and assumes an expanded configuration encircling the catheter 30, balloon 32 and stent 34. This step of the method is illustrated in FIG. 6B.

Referring to FIG. 6C, once distal region 20 is deployed from sheath 12, catheter 30 and ostial locator device 10 then are advanced over guidewire 40 until balloon 32 and stent 34 enter branch vessel BV. Because the diameter of expanded section of distal region 20 is greater than the diameter of the ostium of branch vessel BV, the expanded section of distal region 20 will not pass through ostium O. Instead, distal region 20 flattens out as it is urged against the tissue surrounding ostium O. As this occurs, the clinician will sense the increased resistance to advancement of catheter 30 and ostial locator device 10, and informing the clinician that the stent is properly positioned. In this manner, the precise location of the stent relative to the ostium of the branch vessel may be determined.

When conducted under fluoroscopic guidance, the clinician also will be able to visually verify the stent placement by observing that the compression of the expanded section of distal region 20. Because the distal region preferably includes a radiopaque feature, the clinician will be able to verify the stent placement without repeated injections of contrast solution. As depicted in FIG. 6C, as the expanded section of distal region 20 flattens, it becomes substantially perpendicular to stent 34, and is expected to be readily visible under fluoroscopic examination.

Referring finally to FIG. 6D, once the precise location of the stent relative to the ostium of the branch vessel has been established, the stent is deployed and catheter 30 and ostial locator device are withdrawn. Since the ostium was properly located, stent is correctly positioned over lesion L.

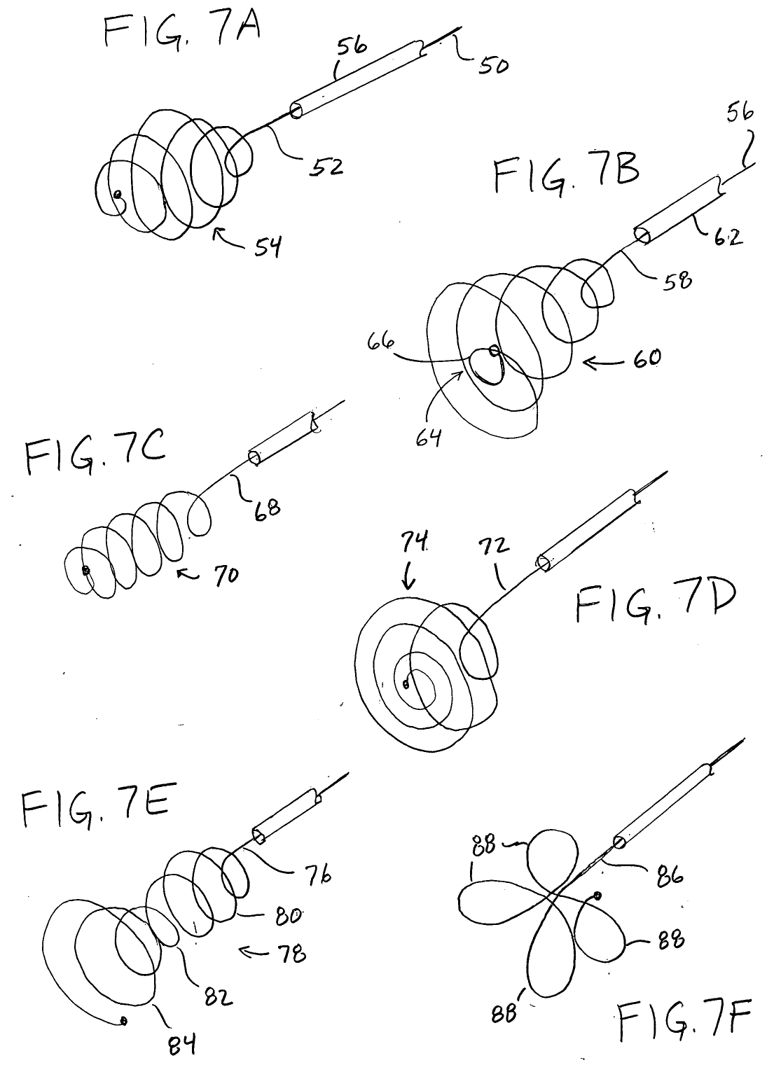

Referring to FIGS. 7A to 7F, several alternative embodiments of the shape assumed by distal region 20 in the expanded configuration are described. In FIG. 7A, ostial locator wire 50 is manufactured and treated using methods that are per se known so that distal region 52 assumes spherical shape 54 when extended beyond the distal end of sheath 56. Preferably, the distal-most turn or turns of ostial locator wire 50 has a diameter that approximates the diameter of the shaft of the interventional device with which the ostial locator wire is to be used, to ensure that spherical shape 54 remains centered on the shaft when it is compressed to locate the vessel ostium.

In FIG. 7B, ostial locator wire 56 is made so that distal region 58 assumes conical shape 60 when extended beyond the distal end of sheath 62. Whereas the embodiment of FIG. 7A includes reduced diameter distal-most turns to retain the expanded section centered during compression, distal region 58 of the embodiment of FIG. 7B includes lasso 64 that forms loop 66. Loop 66 of lasso 64 is designed to be placed over the interventional device prior to affixing sheath 62 to the shaft of the interventional device, and retains conical shape 60 centered on the interventional device during compression and location of the branch vessel ostium. The reduced diameter distal-most turns of the embodiment of FIG. 7A and the lasso of the embodiment of FIG. 7B may be used in conjunction with any of the shapes described herein with respect to the embodiments of FIGS. 7A-7E.

Referring now to FIG. 7C, distal region 68 forms elongate coil 70, while in the embodiment of FIG. 7D, most of distal region 72 forms an approximately flat spiral disk 74. In the embodiment of FIG. 7E, distal region 76 forms vase-like or amphora shape 78, in which having body portion 80, neck region 82 and mouth 84. In the alternative embodiment of FIG. 7F, distal region 86 forms four spaced-apart petals 88. As will of course be understood by one of skill in the art of shape memory alloy manufacture, the ostial locator wire of the present invention may be made to assume any of a myriad of shapes, so long as the wire encircles the interventional device when deployed from its sheath.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

1. Apparatus for locating an interventional device relative to the ostium of a branch vessel, comprising:
a sheath having proximal and distal ends, and a lumen extending therebetween, the sheath adapted to be affixed to an interventional device;
an ostial locator wire slidably disposed within the sheath, the ostial locator wire having a distal region that assumes an expanded configuration when extended from the distal end of the sheath and partially encircles the interventional device.

2. The apparatus of claim 1, further comprising a fastener for affixing the sheath to the interventional device.

3. The apparatus of claim 2, wherein the fastener comprises a thin flexible sheet configured to wrap around the interventional device.

4. The apparatus of claim 2, wherein the fastener comprises a clasp.

5. The apparatus of claim 4, wherein the clasp is adapted to be snap-fit or friction-fit into engagement with the interventional device.

6. The apparatus of claim 4, wherein the clasp is adapted to be affixed to the interventional device using a biocompatible adhesive.

7. The apparatus of claim 1, wherein the expanded configuration has a diameter larger than a diameter of the ostium of the branch vessel.

8. The apparatus of claim 7 wherein a section of the distal region that assumes the expanded configuration assumes a spiral shape.

9. The apparatus of claim 7 wherein a section of the distal region that assumes the expanded configuration defines a portion of a disk, coil, sphere, cone, amphora or petalled-arrangement.

10. The apparatus of claim 1, wherein the ostial locator wire further comprises an atraumatic tip.

11. The apparatus of claim 1, wherein the ostial locator wire further comprises a tip having a lasso that assists in retaining the expanded configuration centered on the interventional device.

12. The apparatus of claim 8, wherein the interventional device is a stent delivery catheter includes a stent, and the spiral shape at least partially encircles the stent.

13. The apparatus of claim 1, wherein the expanded configuration flattens out upon being urged into contact with tissue surrounding the ostium of the branch vessel.

14. The apparatus of claim 12 wherein a distal-most turn of the expanded configuration has a diameter substantially the same as a diameter of the interventional device encircled by the distal region, so as to retain the expanded configuration centered on the interventional device.

15. The apparatus of claim 1, wherein the distal region further comprises a radiopaque feature.

16. A method of locating an interventional device relative to the ostium of a branch vessel, comprising:
providing an interventional device;
providing an ostial locator device having an ostial locator wire;
attaching the ostial locator device to the interventional device so that an expandable section of a distal region of the ostial locator device is disposed at a selected location relative to a distal end of the interventional device;
advancing the ostial locator device and interventional device through a main vessel until the distal end of the interventional device is disposed in the vicinity of the branch vessel;
extending the ostial locator wire so that the expandable section of the ostial locator wire deploys to partially encircle the interventional device;
advancing the interventional device so that the distal end of the interventional device enters the branch vessel and the expandable section flattens out against tissue surrounding the ostium of the branch vessel.

17. The method of claim 16, wherein attaching the ostial locator device to the interventional device comprises applying a fastener to couple the ostial locator device to the interventional device.

18. The method of claim 17 wherein applying a fastener comprises wrapping a thin flexible sheet around the ostial locator device and the interventional device.

19. The method of claim 17 wherein applying a fastener comprises applying a clasp that engages the ostial locator device to the interventional device.

20. The method of claim 16, wherein extending the ostial locator wire so that an expandable section of the ostial locator wire deploys to partially encircle the interventional device comprises extending the ostial locator wire so that a diameter of the expandable section is larger than a diameter of the ostium of the branch vessel.

21. The method of claim 16, wherein providing an ostial locator device having an ostial locator wire further comprises providing an ostial locator device having an ostial locator wire with a radiopaque feature.

22. The method of claim 20 further comprising, during advancing the interventional device so that the distal end of the interventional device enters the branch vessel and the expandable section flattens out against tissue surrounding the ostium of the branch vessel, sensing an increase in resistance to further advancement of the interventional device.
